(19) United States
(12) Patent Application Publication
(10) Pub. No.: US 2026/0364078 A1
(43) Pub. Date: Nov. 19, 2026

(54) PURINE NUCLEOSIDE PHOSPHORYLASE INHIBITORS FOR THE TREATMENT OF METABOLIC SYNDROME AND ASSOCIATED CONDITIONS

(71) Applicant: METASHAPE PHARMA AG, Riehen (CH)

(72) Inventors: Thomas Mehrling, Riehen (CH); Shanta Bantia, Riehen (CH)

(21) Appl. No.: 19/138,814

(22) PCT Filed: Dec. 13, 2023
(86) PCT No.: PCT/EP2023/085709

§ 371 Date: Jun. 13, 2025

(30) Foreign Application Priority Data
Dec. 13, 2022 (GB) 2218782.7

(51) Int. Cl. A61K 31/519; A61K 31/7068; A61K 45/06; A61P 3/04

(52) U.S. Cl. CPC A61K 31/519; A61K 31/7068; A61K 45/06; A61P 3/04

(57) ABSTRACT

PNP inhibitors useful in the treatment of and/or prevention of metabolic syndrome and metabolic syndrome-induced diseases and conditions are provided. PNP inhibitors are also useful in the treatment of and/or prevention of diseases associated with decline of nicotinamide dinucleotide (NAD+). Specific preferred salts for use in these approaches and dosing schedules that avoid immunogenic side effects of these compounds are also disclosed.

### CROSS REFERENCE TO RELATED APPLICATIONS

This application is a U.S. National Stage Application filed under 35 U.S.C. § 371, based on International Patent Application No. PCT/EP2023/085709, filed on Dec. 13, 2023, which claims priority to UK Patent Application No. GB2218782.7, filed Dec. 13, 2022. The entire contents of each of the above applications are incorporated herein by reference.

### FIELD OF THE INVENTION

This disclosure relates to the use of Purine Nucleoside Phosphorylase (PNP) inhibitors such as ulodesine and salts thereof, in the treatment and/or prevention of metabolic syndrome and diseases and conditions that are induced by it; and in the treatment or prevention of diseases and conditions associated with decline of the metabolite, NAD+. The disclosure also further identifies and characterises the preferred salt forms of ulodesine and pharmaceutical compositions for use in such treatments and preventions.

This disclosure particularly describes use of ulodesine hemiglutarate, and pharmaceutical compositions comprising the salt in the treatments and preventions disclosed.

Particularly the inventors have identified that treatment of subjects with a PNP inhibitor such as ulodesine and its salts, leads to a significant increase in both inosine and NAD+ in tissues. As detailed further herein this result leads directly to potential treatment and/or prevention for a variety of diseases and conditions including those associated with a decline in tissue levels of the metabolite, NAD+.

In aspects, this disclosure therefore relates to pharmaceutical methods, compounds and compositions for the treatment and/or prevention of metabolic syndrome and metabolic syndrome induced disease.

### BACKGROUND TO THE INVENTION

Metabolic syndrome is a cluster of conditions that occur together, increasing risk of heart disease, liver disease, stroke and type 2 diabetes. This cluster of conditions includes increased blood pressure, high blood sugar, excess body fat, particularly around the waist, fatty liver and abnormal cholesterol or triglyceride levels (dyslipidemia).

Recently agents, such as glucagon-like peptide 1 (GLP-1) agonists (typically provided in an injectable protein form) have shown some promise in addressing some aspects of metabolic syndrome. These compounds act in the brain to suppress appetite but rebound weight gain is common once treatment is stopped. Patients feel nauseous and reduce food intake during active use of the drug which leads to weight loss of approximately 15%. Reducing blood sugar and stimulating the secretion of insulin causes reduced energy consumption and upon cessation of this form of therapy, patients weight rebounds possibly because food intake is increased and fat cells shrink but do not disappear.

There is now a body of evidence that the actions of brown adipose tissue (BAT) or the browning (conversion) of white adipose tissue (WAT) can protect against obesity (Harms M. and Seale P., Nature Medicine, 2013, 19:1252-1263; Bartelt A. and Heeren J., Nature Reviews Endocrinology, 2014, 10:24-36).

The molecular mechanisms that are responsible for the energy-dissipating qualities of brown fat have been studied in detail (Cannon B. and Nedergaard J., Physiological Reviews, 2004, 84:277-3594; Rosen E. D. and Spiegelman B. M., Cell, 2014, 156:20-44). Uncoupling protein-1 (UCP-1) has been identified as the key factor controlling the thermogenic capacity of brown adipocytes (Klingenberg, Journal of Bioenergetics and Biomembranes, 1999, 31:419-430). UCP-1 disrupts the electrochemical gradient across the mitochondrial membrane by allowing protons to re-enter the mitochondrial matrix. Consequently, mitochondrial fatty acid oxidation is increased and chemical energy is “wasted” through heat production (known as “adaptive thermogenesis”). This process has long been believed to occur exclusively in brown and brown-like (beige) adipocytes.

It has been previously reported that treatment of healthy subjects with PNP inhibitors lead to increased levels of inosine (Viegas et al., J. Clin. Pharmacol 2000, 40: 410-420). Recently, it was discovered that inosine (a purine nucleoside) stimulates energy expenditure in brown adipocytes via the cAMP/protein kinase A signalling pathway. Treatment of mice with inosine increased BAT dependent energy expenditure and induced browning of white adipose tissue tissue also called beige adipose tissue.

Diet induced obese mice treated with inosine daily for 26 days demonstrated significant reduction in weight compared to vehicle Further, inosine is released during apoptosis of brown fat cells and may have a “replace me” signalling function that regulates thermogenic fat and has the potential to counteract obesity in mice. (Niemann et al., Nature, 2022, 609: 361-368).

As the amount of metabolically active BAT seems to be particularly low in patients with obesity or diabetes mellitus, it would be desirable to identify therapies that would increase the production of BAT and/or increase the capacity for adaptive thermogenesis through activation of BAT in these patients.

NAD+ levels steadily decline during aging. By the time a mouse or human is middle aged, levels of NAD+ have fallen to half of youthful levels, with resulting loss of sirtuin (SIRT) activity. Several studies in recent years have shown that treatment of old mice with poly (ADP-ribose) polymerase (PARP) inhibitors (which increase NAD+ levels), with NAD+ precursors or with inhibitors of NAD+ degradation can greatly improve health. Observed effects include increased insulin sensitivity, decreased cholesterol and triglyceride levels, decreased weight gain, reduced stem cell senescence, and extension of lifespan. (Bai et al., Cell Metab. 2011, 13:461-468; Gomes et al., Cell. 2013, 155:1624-1638; Yoshino et al., Cell Metab. 2011, 14:528-536; Zhang et al., Science. 2016, 26:2016).

Improvement of these metabolic parameters essentially leads to “healthy longevity”, an effect that has been demonstrated with NAD+ precursors and also with inhibitors of NAD+ degraders such as CD38 (Peclat et al., Aging Cell. 2022, 21:e13589). Genetic mouse models with altered NAD+ biosynthesis phenotypes show similar results.

Non-alcoholic fatty liver disease (NAFLD) is the hepatic manifestation of metabolic syndrome, and covers a spectrum of conditions, from simple steatosis, where lipid accumulates in the liver, to non-alcoholic steatohepatitis (NASH) characterized by fibrosis, inflammation and hepatocyte cell death. If left unchecked, NASH can progress to end-stage liver diseases such as cirrhosis and hepatocellular carcinoma (HCC). One goal of effectively treating NAFLD and NASH is therefore to prevent or reduce the likelihood of these conditions progressing to cirrhosis and HCC. The prevalence of NAFLD is increasing worldwide, and has reached epidemic proportions. Estimates of NAFLD prevalence suggest that up to 1 billion people might be affected worldwide (Loomba R, Sanyal AJ. Nat Rev Gastroenterol Hepatol. 2013 November; 10(11):686-9). Hence, there is a need for novel treatments that can be administered in a safe and cost-efficient manner to treat these conditions.

Genetic and interventional models in mice suggest that modulating the NAD+ synthetic pathway has the potential to ameliorate fatty liver disease For example, treatment with NAD+ precursors has been suggested to prevent the progression of NAFLD to NASH and NAD+ precursor supplementation is associated with decreased hepatic stellate cell activation, and decreased fibrosis. (Dall et al., J Physiol. 2022, 600.5: 1135-1154). Furthermore, sirtuins, which cleave NAD+ to nicotinamide (NA) and 0-acetyl-ADP-ribose, may also play a role in NAFLD. Decreased hepatic expression of SIRT1, SIRT3, SIRT5, and SIRT6 has been reported in patients with NAFLD (Wu et al Ann Clin Lab Sci. 2014; 44(4):410-8), while liver-specific knockout of SIRT1 and SIRT6 in mice results in hepatic lipid accumulation (Kim et al., Cell Metab. 2010; 12(3):224-36; Purushotam et al., Cell Metab. 2009; 9: 327-338). Thus overexpression of SIRT1 appears to protect against high fat diet (HFD)-induced obesity (Pfluger et al., Proc Natl Acad Sci USA. 2008; 105(28):9793-8), and increasing the hepatic NAD pool by inhibition of NAD+-consuming PARPs decreases weight gain and hepatic steatosis development by a SIRT1-dependent mechanism in high-fat high-sucrose-fed mice (Gariani et al., J Hepatol. 2017; 66(1):132-141). Hence, sufficient sirtuin activity appears important for prevention of hepatic lipid accumulation.

PNP inhibitors have been available for many years. Example compounds are described in U.S. Pat. Nos. 5,565,463, 5,008,270, 5,985,848, 7,109,331, 7,553,839, 7,427,624, WO08030119A1, U.S. Pat. No. 8,283,345, CZ20190667A3, and CZ34625U1 all of which are incorporated herein by reference. In particular U.S. Pat. No. 7,553,839 describes the synthesis of ulodesine (see compound 8). These documents also describe methods of manufacturing the compounds and various formulations comprising these molecules.

PNP inhibitors such as ulodesine and forodesine, and well known salts of ulodesine, such as succinate, have previously been suggested as urate lowering therapies in the treatment of conditions such as gout, and hyperuricemia because of their ability to inhibit uric acid synthesis through inhibition of the synthesis of both hypoxanthine (from inosine) and guanine (from guanosine), each of which is mediated by PNP. Ulodesine has also been proposed as a novel immune check point inhibitor in the treatment of leukemia due to its indirect effect on TLR7 activation, through increasing the pool of (deoxy)guanosine ligands (Abt, et al. J. Clin. Invest. 132, e1608 (2022). PNP inhibitors such as ulodesine have also been proposed as compounds for lowering dNTP pools in the treatment of cancer (WO21022105A1). There is no teaching in the literature that PNP inhibitors may be used to treat or prevent metabolic syndrome or its associated conditions and diseases.

There remains a need to provide novel therapeutic solutions for the treatment and prevention of, metabolic syndrome and the related disorders and diseases such as obesity, fatty liver diseases and cardio-metabolic disorders such as heart disease, stroke and type 2 diabetes. The potential for a PNP inhibitor to have therapeutic relevance in these conditions, was not identified, hypothesised or practically investigated until the present work of the applicant. It may now be inferred from the results provided and with regard to the mechanisms now considered essential at the cellular level, that practical use of such a PNP inhibitor or particular stable salt thereof, as defined herein, such as ulodesine, would be extremely beneficial in these therapeutic indications.

### SUMMARY OF INVENTION

The present inventors have identified, for the first time, that inhibition of PNP (for example using ulodesine or a salt thereof) leads to significant modulation of metabolic indicators including weight gain, % wt of brown and white adipose tissue, fatty acid synthesis, liver triglycerides, cholesterol, blood glucose and plasma insulin, as well as lowering serum aspartate aminotransferase (AST) and alanine transaminase (ALT), and liver AST. PNP inhibitors may therefore be used to treat a number of conditions including metabolic syndrome and its associated diseases and conditions as discussed further herein.

The preferred inhibitor of PNP comprises a glutarate salt of ulodesine. In embodiments, the salt comprises a hemi-glutarate salt of ulodesine. Until very recently, it was a technical challenge to identify and produce reliably a stable form of ulodesine and which would be suitable for application in pharmaceutical production. A physically stable salt is a highly desirable property in pharmaceutical manufacturing and is therefore of particular interest in medical applications and methods concerning ulodesine which are disclosed and discussed herein.

PCT application, published as WO2023001893, successfully produced ulodesine hemi-glutarate for the first time, using a new process and generating a stable new salt that could be useful in pharmaceutical development. For the avoidance of doubt the content of that publication, as it relates to ulodesine hemi-glutarate and its method of production, is incorporated by reference herein.

The authors of that application noted typical salt variants made by routine methods in the art did not consistently produce reliably stable compounds, which is required for the upscale manufacture of ulodesine in pharmaceutical development. Methods available (in the general knowledge) and those known in the art for making other existing salts of ulodesine had been attempted by the authors but these had not been useful for the successful preparation of the glutarate salt. For this reason, as a starting point, as compared to other salts available at the time, the glutarate salt was not an obvious candidate to try.

Furthermore, salt selection requires consideration of several analytical parameters to determine helpful chemical and physical properties, such as clear and sharp diffraction peaks in graphics, any obvious amorphous peaks, solvent weight loss and the ability to obtain a crystal form under a variety of conditions. Glutaric acid was not a practical choice for use in the production of ulodesine under the standard criteria since initial analysis described by the authors indicated low crystallinity and some obvious solvent weight loss of the product when its production was attempted using the known methods.

Nonetheless, having extensively researched and developed alternative means of production in an attempt to overcome the challenges, a novel process for making hemi-glutarate was identified and this produced a surprisingly highly stable crystal salt, as compared to other salts. In embodiments, the ulodesine hemi-glutarate for use in the present invention may be made by that same process, comprising the steps of: (a) preparing a solution of ulodesine free base in water, and optionally stirring at room temperature; (b) adding glutaric acid to the mixture of step (a), and optionally stirring for 30 min at room temperature; (c) freeze-drying the solution of step (b) to yield a white solid product; (d) dissolving the solid product of (c) in water; optionally heating up to 75 °C; adding ethanol and optionally stirring at 75 °C for 30 minutes, to form a homogeneous solution; (e) making a dropwise addition of acetonitrile to the solution of (d), optionally over a period of 60 minutes; (f) stirring the solution of (e) for 60 minutes, optionally at 75 °C; and optionally cooling the solution to 0 °C over a period of 60 minutes; (h) Filtering and washing with acetonitrile to obtain ulodesine hemi salt glutarate salt.

The chemical structure of ulodesine hemi-glutarate is provided below:

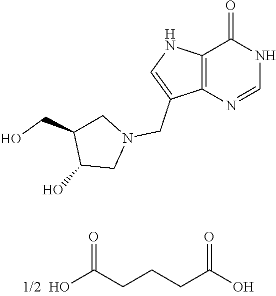 [SMILES: O=c1[nH]cnc2c(CN3C[C@H](CO)[C@@H](O)C3)c[nH]c12 ; O=C(O)CCCC(=O)O]

In the methods, glutaric acid was added with an amount of the desired final salt form to assist and optimise the crystallization process. The specific recrystallization steps identified above and the selection of solvent and number, were critical to obtain a hemi salt ethanol (in conjunction with other steps) i.e. the use of ACN (acetonitrile)/water for the formation of the final hemi glutarate crystal, otherwise less favourable salts, such as mono salts, were obtained.

The present application provides further analytical work herein, which confirms the recrystallisation process and hemi glutarate salt form selected, its stability and thus suitability for the medical uses recited herein. This additional information is provided in the detailed description of the invention disclosure and the figures that accompany that disclosure.

In embodiments, the ulodesine hemi-glutarate is the stable crystal form type A as defined clearly by the data inclusion herein. The experimental evidence provides for a stable and well characterised crystal structure of ulodesine hemi-glutarate.

In one aspect the present disclosure therefore provides a PNP inhibitor for the treatment or prevention of metabolic syndrome or a metabolic syndrome-induced disease or condition. The term “PNP inhibitor” includes those compounds that inhibit PNP. Compounds having in-vitro inhibitory constant (Ki) values of less than about 5×10-7 M, typically less than about 1×10-8 M, and preferably less than 5×10-9 M are preferred for in vivo use.

In some embodiments the disclosure provides the use of a PNP inhibitor in the manufacture of a medicament for the treatment of metabolic syndrome and metabolic syndrome induced conditions.

As mentioned above, metabolic syndrome is a cluster of conditions that tend to occur together. This cluster of conditions includes increased blood pressure, high blood sugar, excess body fat, particularly around the waist (i.e abdominal fat), fatty liver and abnormal cholesterol or triglyceride levels (dyslipidemia).

Abnormal cholesterol or triglycerides includes high fasting serum LDL cholesterol, low fasting serum HDL cholesterol and high fasting serum triglycerides compared to normal subjects.

Reference to treatment or prevention of metabolic syndrome herein relates to treatment or prevention of at least one, at least two, at least three or more of the underlying conditions mentioned.

In one embodiment treatment or prevention of metabolic syndrome herein includes at least one preferably at least two of the following: treatment or prevention of high blood pressure, treatment or prevention of insulin resistance, normalisation of blood glucose, a reduction in body fat (e.g. waist fat), an increase in brown fat as a percentage of body weight, treatment or prevention of fatty liver and/or a normalisation of abnormal serum cholesterol and/or triglycerides.

In one embodiment treatment or prevention of metabolic syndrome herein includes at least one preferably at least two of the following: a reduction in high blood pressure, a reduction in insulin resistance, a normalisation of blood glucose, a reduction in body fat (eg waist fat), an increase in brown fat as a percentage of body weight, a reduction in fatty liver, a normalisation of abnormal serum cholesterol and/or triglycerides.

In one embodiment treatment or prevention of metabolic syndrome includes at least a reduction in insulin resistance and a normalisation of blood glucose; and optionally at least one of: a reduction in high blood pressure, a reduction in body fat (eg waist fat), an increase in brown fat as a percentage of body weight, a reduction in fatty liver and a normalisation of abnormal serum cholesterol and/or triglycerides.

In one embodiment treatment or prevention of metabolic syndrome includes at least a normalisation of serum cholesterol and a reduction in body fat (e.g. waist fat); and optionally at least one of: a reduction of insulin resistance, a normalisation of blood glucose, a reduction in high blood pressure, an increase in brown fat as a percentage of body weight, a reduction in fatty liver and a normalisation of serum triglycerides.

In one embodiment treatment or prevention of metabolic syndrome includes at least a normalisation of serum cholesterol and reduction of insulin resistance and optionally at least reduction in body fat normalisation of blood glucose, a reduction in high blood pressure, an increase in brown fat as a percentage of body weight, a reduction in fatty liver and a normalisation of serum triglycerides.

Reduction in insulin resistance means a reduction in the HOMA-IR score as detailed below.

Normalisation of blood glucose means lowering fasting blood glucose concentrations such that it is closer to levels found in subjects who do not suffer from metabolic syndrome.

Normalisation of abnormal serum cholesterol and/or triglycerides means one or more of lowering total fasting serum cholesterol, lowering fasting serum LDL (low-density lipoprotein) cholesterol and/or lowering fasting serum triglycerides, such that the levels are is closer to levels found in subjects who do not suffer from metabolic syndrome.

Without wishing to be bound by any theory, the inventors believe the mechanism behind this effect is the following: Inosine is broken down by purine nucleoside phosphorylase (PNP) to hypoxanthine and when this enzyme is inhibited the levels of inosine rises. In the salvage pathway to inosine monophosphate (IMP) synthesis, hypoxanthine guanine phosphoribosyl transferase (HGPRT) converts inosine to IMP, and in doing so consumes phosphoribosyl diphosphate (PRPP). HGPRT is normally a major consumer of PRPP and when PNP is inhibited, hypoxanthine and guanine are not produced, essentially sparing PRPP (FIG. 1, FIG. 2).

Normally, the rate of conversion of nicotinamide (NAM) and nicotinic acid (NA) to NAD+ is limited by the availability of PRPP. Inhibition of PNP however, leads to a larger pool of available PRPP and this leads, in turn, to the significantly increased NAD+ level in whole blood identified in experiments detailed further herein.

In addition to the above route, it is notable that the NAD+ precursor, nicotinamide riboside (NR), is metabolised through PNP. Thus inhibition of PNP may lead to a larger pool of NAD+ precursors and a consequent increase in NAD+ levels.

The cluster of conditions referred to as metabolic syndrome together or individually can lead to more severe conditions or diseases such as heart disease, liver disease, stroke and type 2 diabetes. Treating or preventing parameters of metabolic syndrome can prevent the underlying conditions progressing, thus addressing metabolic syndrome as described herein can lead to prevention (or a reduced risk of) peripheral vascular disease, cardiovascular disease, such as heart disease and stroke, and prevent or treat type 2 diabetes, prevent or treat liver diseases including fatty liver diseases such as NASH or NAFLD and prevent or treat dyslipidemia.

Dyslipidemia refers to an imbalance of lipids such as cholesterol (i.e. total serum cholesterol), serum low-density lipoprotein cholesterol, (LDL-C), serum high-density lipoprotein cholesterol (HDL-C) and serum triglycerides. In one embodiment treatment with a PNP inhibitor may prevent or treat dyslipidemia. In one embodiment treatment with a PNP inhibitor may one or more or all of these effects: lower serum LDL cholesterol, raise serum HDL cholesterol, lower serum total cholesterol and/or lower total serum triglyerides. In another embodiment treatment with a PNP inhibitor may lower serum total cholesterol. In another embodiment treatment with a PNP inhibitor may lower serum triglycerides.

Diabetes is a chronic, metabolic disease characterized by elevated levels of blood glucose, which leads over time to serious damage to the heart, blood vessels, eyes, kidneys and nerves. Type 2 diabetes usually occurs in adults, as a result of insulin resistance or where insulin production becomes reduced. Insulin resistance is a condition which is closely linked to metabolic syndrome and occurs in the majority of patients with metabolic syndrome. In insulin resistance cells (eg those in muscle, fat, and liver) fail to respond to insulin by increasing the uptake of glucose. Under these conditions blood glucose is increased and, due to higher levels of secretion by the pancreas, insulin levels may also be high. A simple measure of insulin resistance is the Homeostasis Model Assessment of Insulin Resistance (HOMA-IR) test.

HOMA-IR=[fasting insulin (μIU/mL)]×[fasting glucose (mmol/L)]/22.5

A HOMA-IR score of <1.0 is optimal, >1.9 indicates early insulin resistance and >2.9 indicates significant insulin resistance.

In some embodiments treatment with a PNP inhibitor can reduce fasting blood glucose in a subject and/or reduces fasting blood insulin in a subject. In some embodiments treatment with a PNP inhibitor can treat or prevent type 2 diabetes in a subject. In some embodiments treatment with a PNP inhibitor can treat or prevent insulin resistance in a subject. In some embodiments treatment with a PNP inhibitor can lower the HOMA-IR score in a subject

Obesity is a condition in which excess body fat has accumulated to such an extent that it can have negative effects on health. The WHO defines overweight as a body mass index (BMI) of 25 or higher, and obese as a BMI of 30 or higher. The U.S. CDC subdivides obesity with class 1 obesity as being a BMI 30 to 35; class 2 obesity as being a BMI of 35 to 40, and class 3 obesity as having a BMI of over 40. In some embodiments treatment with a PNP inhibitor can lower the weight of a subject. In some embodiments treatment with a PNP inhibitor can prevent or treat weight gain in a subject and in some embodiments treatment with a PNP inhibitor can prevent or treat obesity and/or overweight in a subject.

Fatty liver diseases such as NAFLD range from simple hepatic steatosis to non-alcoholic steatohepatitis (NASH). In some embodiments, treatment with a PNP inhibitor can prevent or treat fatty liver diseases, in other embodiments treatment with a PNP inhibitor can prevent or treat NAFLD. In some embodiments treatment with a PNP inhibitor can prevent or treat NASH. In some embodiments, treatment with a PNP inhibitor can reduce hepatic steatosis.

Improving metabolic syndrome has been shown to lower the incidence of cardio-metabolic diseases such as cardiovascular conditions such as heart disease; stroke and type 2 diabetes (Wilsone et al., Circulation 2005; 112: 3066-3072) and therefore in some embodiments treatment with a PNP inhibitor can treat or prevent cardiometabolic diseases, in some embodiments treatment with a PNP inhibitor can treat or prevent cardiovascular disease. In some embodiments treatment with a PNP inhibitor can treat or prevent heart disease. In some embodiments treatment with a PNP inhibitor can treat high blood pressure. In some embodiments treatment with a PNP inhibitor can lower blood pressure. In some embodiments treatment with a PNP inhibitor can treat or prevent stroke.

Many PNP inhibitors are known. Example compounds are described in U.S. Pat. Nos. 5,565,463, 5,008,270, 5,985,848, 7,109,331, 7,553,839, 7,427,624, WO08030119A1, U.S. Pat. No. 8,283,345, CZ20190667A3, CZ34625U1 all of which are incorporated herein by reference. In particular U.S. Pat. No. 7,553,839 describes the synthesis of ulodesine (see compound 8). These documents also describe methods of manufacturing the compounds and various formulations comprising these molecules.

Further compounds are known from the literature, for example, transition state analogues are described in Evans et al., Organic Letters, 2003, 5:3639; Taylor et al., Journal of American Chemical Society, 2007, 129:6984; Evans et al., Journal of Medicinal Chemistry, 2003, 46:5271; Castilho et al., Bioorganic and Medicinal Chemistry, 2006, 14:516; Schramm et al., Journal of Biological Chemistry, 2007, 282:28297; and Bantia et al., International Immunopharmacology, 2010, 784 and 2001, 1:1199-1210; Kicska et al., Proceedings of National Academy of Sciences, 2001, 98:4593-4598; Ho et al., Proc Natl Acad Sci USA. 2010, 107(11):4805-12). The disclosure of each of these references is hereby incorporated in its entirety by reference.

Preferred compounds are those of the formulas I, II and III below.

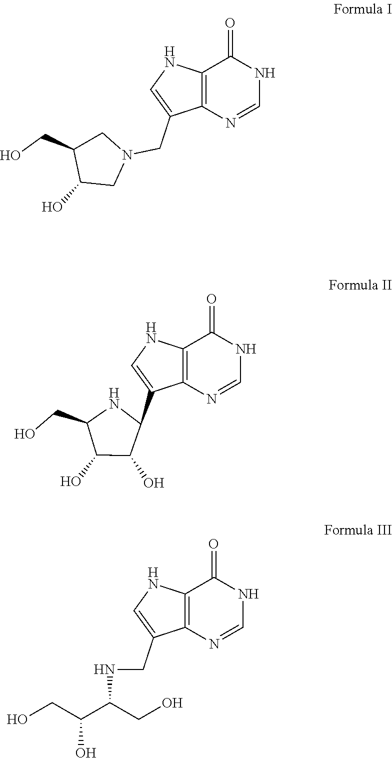 [SMILES: O=c1[nH]cnc2c(CN3C[C@H](CO)[C@@H](O)C3)c[nH]c12 ; O=c1[nH]cnc2c([C@@H]3N[C@H](CO)[C@@H](O)[C@H]3O)c[nH]c12 ; O=c1[nH]cnc2c(CN[C@H](CO)[C@H](O)CO)c[nH]c12]

Preferably, the PNP inhibitor is ulodesine (formula I) or a pharmaceutically acceptable salt thereof. Known salts of ulodesine include hydrochlorides, dihydrochlorides, hydrobromides, hemisulfate, p-tosylate, phosphate, citrate, L-tartrate, L-lactate, stearate, maleate, succinate, fumarate, and L-malate and L-aspartate (see for example WO2010/111381).

The present inventors have identified and further characterised an advantageous salt, the hemiglutarate. The PNP inhibitor is therefore preferably comprises ulodesine hemiglutarate.

In an embodiment, the disclosure provides a pharmaceutical composition comprising the hemiglutarate salt of ulodesine, which optionally comprises a pharmaceutically acceptable carrier, diluent and/or excipient.

The magnitude of a therapeutic dose of PNP inhibitor or a pharmaceutically acceptable salt, solvate, hydrate, or stereoisomer thereof in the acute or chronic treatment will vary, with the nature and severity of the disease and the route by which it is administered. The dose, and in some cases the dose frequency will also vary according to disease treated, the age, body weight and response of the individual patient. The PNP inhibitors of the present disclosure may be used at a dose of between 1 mg and 1000 mg, but more typically at up to 500 mg and yet more typically at up to 250 mg. In some embodiments the dose is 1 mg to 150 mg or 1 mg to 120 mg. in some embodiments the dose is 5 mg to 120 mg. Doses identified herein are those appropriate for an adult 80 kg human.

Inherited deficiencies in PNP, are known to lead to a severe combined immunodeficiency (Markert ML. Immunodefic Rev. 1991; 3(1):45-81). Likewise, it is known that PNP inhibition in humans over long term at high doses leads to decreases in various lymphocyte subsets (Gomes et al., Blood ASH Annual Meeting Abstracts, 2008, 112:Abstract 2583). Hence, long term treatment with high doses of PNP inhibitor given continuously may have immunogenic effects.

However, despite this technical prejudice against the use of these compounds in therapy, the applicant has shown herein that therapeutic solutions may in fact remain a plausible approach.

In order to counteract the immunogenic side effects seen in these drugs, the disclosure provides low and/or intermittent dosing of a PNP inhibitor (such as ulodesine hemi-glutarate), which allows for production and elevation of the endogenous metabolites inosine and NAD+ which are able to facilitate critical changes at a cellular level and lead to attenuation of obesity and metabolic syndrome. This low and/or intermittent dosage exploits the natural endogenous metabolite inosine as a novel means to activate BAT to decrease fat tissue, improving obesity and also exploits the natural endogenous metabolite NAD+ to activate sirtuins and other enzymes that improve metabolic risk factors, but without leading to significant immune side effects, such as reduced lymphocyte levels.

In one embodiment, therefore, the inventors have provided a new regimen for administration of the drug, which provides beneficial therapeutic effects, but with low or minimal side effects. This dosing regimen comprises a further embodiment of the invention.

In some embodiments therefore, this disclosure provides a PNP inhibitor for use as described elsewhere herein wherein the PNP inhibitor is provided in an intermittent dosing regime, a low dosing regime or a combination of the two as described further below.

The PNP inhibitors of the present disclosure may be used at a dose of between 1 mg and 1000 mg/day In one embodiment they are used at a dose of up to 500 mg/day. In one embodiment they are used at a dose of up to 250 mg/day

Where lower dose regimens are required, for example in order to reduce the incidence of immunogenic side effects, doses of 1 mg to 150 mg/day; optionally 1 mg to 100 mg/day; optionally 1 mg to 50 mg/day or 1 mg to 40 mg/day may be provided. In some embodiments the lower dose in these ranges is 10 mg for example 10 mg to 40 mg. Such doses represent the daily dose provided and may be provided in 1, 2, 3 or more sub doses over the day as appropriate. Where dosing targets reduced incidence of immunogenic side effects, dosing targets a <10% or <20% reduction in lymphocyte numbers in blood.

In some embodiments, the intermittent treatment regimen comprises or consists of repeated cycles of periods of treatment wherein the subject receives the daily dose of drug (dosing), followed by break periods wherein the subject receives no drug.

In one embodiment one cycle of treatment comprises or consists of dosing for 1 to 6 days, followed by a break of at least 1 day. In one embodiment one cycle of treatment comprises or consists of dosing for 2 to 4 days, followed by a break of at least 1 day (optionally 2, optionally 3, optionally 4 days). In one embodiment one cycle of treatment comprises or consists of dosing for 3 days, followed by a break of at least 1 day (optionally at least 2, optionally at least 3, optionally at least 4 days). Typically break periods are no longer than 5, 6 or 7 days. In one embodiment, the cycle of treatment comprises or consists of dosing for 2 to 4 days, preferably 3 days, followed by a break of 3 to 4 days.

An intermittent dosing regimen is thought to be sufficient to induce the metabolic changes but critically without significantly impacting the lymphocyte number (<10% or <20% change) and therefore does not have an immunogenic response.

Immunogenic side effects may also be mitigated by treating the subject with both a lower dose and an intermittent dosing regimen, thus in some embodiments, the lower regimen described above may be provided in the intermittent regimen described. Thus in some embodiments a dose of 1 mg to 90 mg may be provided daily in an intermittent dosing regime in which the subject receives repeated cycles of dosing wherein a cycle comprises or consists of dosing for 1 to 6 days, followed by a break of at least 1, at least 2 at least 3 at least 4 and no more than 7, 6 or 5 days.

Cycles may be continued for the period of treatment, or may be varied according to need.

For compliance purposes, it may be useful for the treatment to operate on a 7 day cycle, so in one embodiment, the subject is treated on days 1 to 6 and not treated on day 7, or treated on days 1 to 5 and not treated on days 6 and 7, or treated on days 1 to 4 and not treated on days 5 to 7, or treated on days 1 to 3 and not treated on days 4 to 7, or treated on days 1 and 2 and not treated on days 3 to 7 or treated on day 1 and not treated on days 2 to 7.

By subject, is meant any subject to be treated according to the disclosed treatments. The subject is typically a mammalian subject and particularly a human subject in need of treatment for the condition to be treated but may also be any other subject including, for example companion or farmed animals.

Compositions of the invention may be formulated as oral dosage forms, parenteral dosage form or topical dosage form. In specific embodiments, the oral dosage form may be formulated to provide slow release of the PNP inhibitor.

In some embodiments the present disclosure provides methods for the treatment of subjects suffering from metabolic syndrome or a metabolic syndrome induced disease, disorder or condition comprising administering to the subject a pharmaceutically effective amount of a PNP inhibitor. In some embodiments the PNP inhibitor is provided in an intermittent dosing regime, a low dosing regime or a combination of the two as described elsewhere herein.

In some embodiments the disclosure provides a method of treating a subject suffering from a metabolic syndrome, disorder, disease or condition, [particularly in which it is desirable to increase the amount of extracellular inosine or NAD+], the method comprising administering to a subject in need thereof, a pharmaceutically effective amount of a PNP inhibitor.

In some embodiments, the disclosure provides a method of increasing the percentage of brown adipose tissue (BAT) to Body weight in a subject in need thereof comprising administering to the subject a pharmaceutically effective amount of a PNP inhibitor. In embodiments, the method may increase the percentage of BAT to body weight. In some embodiments the method may increase the percentage volume of BAT to body volume. In some embodiments, the method may increase the overall proportion of BAT to WAT, particularly to iWAT.

In some embodiments the disclosure provides a method of improving the lipid profile in a subject in need thereof comprising administering to the subject a pharmaceutically effective amount of a PNP inhibitor. By improving the lipid profile is meant lowering the plasma concentration of one or more of cholesterol, triglycerides and low density lipoprotein (LDL). Particularly cholesterol.

In some embodiments the disclosure further provides a method of treating a fatty liver disease, in a subject in need thereof the method comprising administering to the subject a pharmaceutically effective amount of a PNP inhibitor. The fatty liver disease may be NASH and/or NAFLD. In some embodiments such treatments have one or more of the following effects: decreasing lipid and/or liver enzyme abnormalities and decreasing steatosis in the liver. In some embodiments the treatment includes lowering liver total cholesterol in the subject.

In some embodiments the disclosure further provides a method of lowering the activity of the liver enzymes AST an/or ALT in a subject in need thereof the method comprising administering to the subject a pharmaceutically effective amount of a PNP inhibitor.

In some embodiments the use of the PNP inhibitor to treat conditions and diseases herein comprises administering the PNP inhibitor according to the low and/or intermittent dose regimen described herein.

In some embodiments, the treatments disclosed herein comprise providing a PNP inhibitor in combination with one or more additional active agents. Examples of other active agents include, but are not limited to, ENT1 inhibitors such as dilazep, GLP-1 agonist (such as exenatide, liraglutide, albiglutide and semaglutide), anti-obesity drugs (such as bupropion-naltrexone), antihypertensives (such as amlodipine, captopril, enalaopril and furosemide) and anti diabetic drugs (such as metformin, rosiglitazone and insulin analogues such as insulin glargine or insulin detamir) or other active agents known in the art.

### FIGURES

FIG. 1 shows a modulation of various purine metabolites through PNP inhibition.

FIG. 2 is a schematic presentation of the role of PNP in purine metabolism, illustrating the possible relationship between PNP inhibition and increases in inosine and NAD+ levels.

FIG. 3 shows the effect of ulodesine hemi glutarate in a Diet Induced Obesity (DIO) mouse model on weight, food intake, body mass blood glucose and serum cholesterol.

FIG. 4 shows the effect of ulodesine hemi glutarate in a DIO mouse model, on serum cholesterol, blood glucose, plasma insulin, serum alanine transaminase (ALT), Serum aspartate transaminase (AST) and percentage of iWAT/body mass.

FIG. 5 shows the effect of ulodesine hemi glutarate in a DIO mouse model on expression of Fatty acid synthesis enzyme markers in iWAT and on thermogenic markers (UPC-1 expression) in BAT tissue.

FIG. 6 shows the effect of ulodesine hemi glutarate in a DIO mouse model on plasma inosine and plasma NAD+ levels.

FIG. 7 shows the effect of ulodesine hemi glutarate on liver parameters (liver total cholesterol (TC) and liver AST) in a DIO mouse model.

FIG. 8 shows the effect of increasing doses of ulodesine hemi glutarate on lymphocytes in Diet Induced Obesity (DIO) mouse model.

The following figures provide the graphic analysis resulting from various analytical testing showing the physical and chemical properties of hemi-glutarate salts produced as disclosed within the context of the present disclosure.

FIG. 10 shows XRPD overlay of Glutarate type A; the scaled up sample and identified as the hemi-glutarate salt of ulodesine, provided in accordance with the present disclosure;

FIG. 11 shows the TGA result and the DSC result of Glutarate type A; the scaled up sample and identified as the hemi-glutarate salt of ulodesine, provided in accordance with the present disclosure;

FIG. 12 shows 1H NMR spectrum of Glutarate type A; the scaled up sample and identified as the hemi-glutarate salt of ulodesine, provided in accordance with the present disclosure

FIG. 13 shows the PLM crystalline structure of Glutarate type A; the scaled up sample and identified as the hemi-glutarate salt of ulodesine, provided in accordance with the present disclosure;

FIG. 14 shows a DVS graphic indicating hygroscopic nature of Glutarate type A; the scaled up sample and identified as the hemi-glutarate salt of ulodesine, provided in accordance with the present disclosure;

FIG. 15 shows a XRPD overlay of the same Glutarate type A before and after the DVS described in relation to FIG. 14;

FIG. 16 shows a XRPD overlay of the drying stability data concerning the same Glutarate type A;

FIG. 17 shows a XRPD overlay characterising Glutarate type A provided from polymorph screening of pre-characterised Ulodesine hemi-glutarate starting (reference) material;

FIG. 18 shows the TGA and DSC data further characterising and confirming the hydroscopic nature of the Glutarate type A provided from a polymorph screening exercise;

FIG. 19 shows the H NMR spectrum further characterising and confirming the stoichiometry of the Glutarate type A provided from a polymorph screening exercise;

FIG. 20 shows an XRPD overlay characterising the Glutarate type B;

FIG. 21 shows an XRPD overlay characterising the Glutarate type C and likely conversion to Glutarate type A; and

FIG. 22 shows an XRPD overlay characterising the Glutarate type C and likely conversion to Glutarate type A.

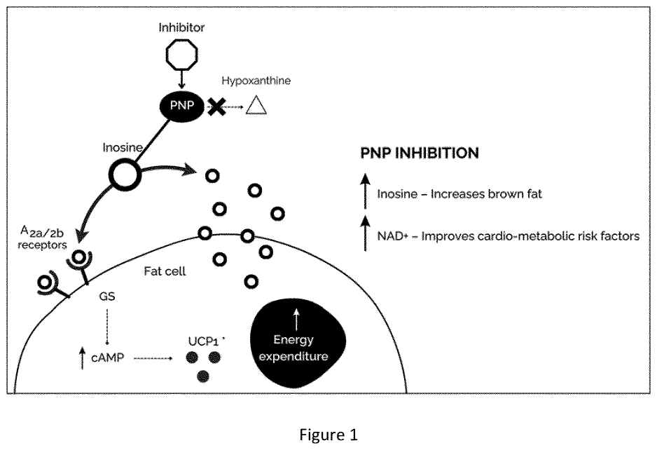

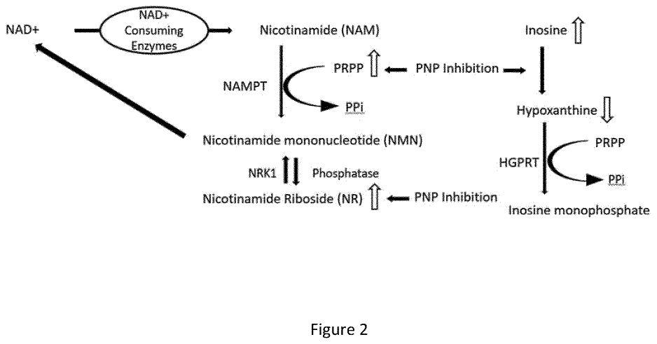

### HEMI-GLUTARATE SALT CONFIRMATION AND CHARACTERISATION

In WO2023001893 the authors disclosed that following several technical variations of the crystallisation process investigated, an alternative re-crystallisation method was required for production of the hemi salt. The chemical structure of ulodesine hemi-glutarate is provided below:

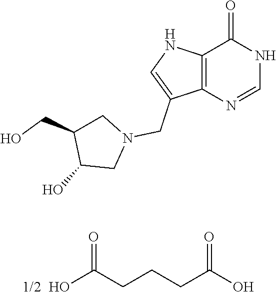 [SMILES: O=c1[nH]cnc2c(CN3C[C@H](CO)[C@@H](O)C3)c[nH]c12 ; O=C(O)CCCC(=O)O]

Using a mixed solvent process, using several different solvents, a new crystallisation method specific to the hemi-glutarate form was determined and is repeated here for clarity: to a solution of Ulodesine (1000.00 mg, 3.78 mmol) in water (50 mL) glutaric acid (278.36 mg, 1.89 mmol) was added. The mixture was stirred at room temperature for 30 min and then lyophilized to give 1155.00 mg of white solid. The yield was 90%, 1055 mg of Ulodesine hemi glutarate salt was dissolved in 3 mL of water and then heated to 75 °C, 15 mL of ethanol was added and stirred at this temperature for 30 min to form a homogeneous solution. Then 30 mL of acetonitrile was added dropwise over 1 h. Then the mixture was stirred at this temperature for 1 h. The mixture was cooled to 0 °C over 1 h. The mixture was filtered and the filter cake was washed by acetonitrile and dried to give 810 mg of Ulodesine hemi glutarate as white solid. The yield was 76.8%.

Analysis determined this solid was crystal form hemi glutarate and confirmed that a new alternative method, including several steps and the addition of ethanol, was necessary for effective recrystallization of Ulodesine hemi glutarate.

| Item | Result |
|---|---|
| Yield | 76.8% |
| LC-MS | 100% purity (214 nm), [M + H]⁺ = 265.0, Ret: 0.42 min. |
| ¹H NMR | Consistent and clean; |
| | 1H NMR (400 MHz, DMSO-d6): δ 11.98 (s, 1H), 11.83 (s, |
| | 1H), 7.79 (s, 1H), 7.27 (s, 1H), 4.80 (br, 1H), 3.78-3.74 |
| | (m, 1H), 3.67-3.58(m, 2H), 3.43-3.40(m, 2H), 3.33-3.21(m, |
| | 2H), 2.72-2.68(m, 2H), 2.49-2.30 (m, 3H), 1.97-1.96 (m, |
| | 1H), 1.92-1.88 (m, 1H). |

Furthermore, it was noted during this work by the authors of that application that it was critical to use the free-base product to produce the desired salt in order to confirm that a complete method of manufacture disclosed resulted in the same useful salt product (rather than starting from the simple free base of ulodesine). If freebase (rather than HCL) was used as the reaction partner, the addition of a base subsequently in the reaction was not necessary. The reaction was more efficient and resulted in higher product content than the previously used mechanism. Importantly, this change also avoided the hydrochloride salt dissociation previously seen in those prior methods. Ulodesine freebase using this method was obtained with high HPLC purity and analytical testing in the application confirmed that, by modifying this earlier step, reliable and consistent production of the hemi-glutarate salt from Ulodesine was possible in a complete method of Ulodesine manufacture.

As seen below, the ulodesine hemi-glutarate batch obtained by the new method was in good yield and verified to be chemically pure. The characteristics of the salt produced were consistent with original salt selection studies in WO23001893.

| Batch No. | Amount | HPLC purity | e.e | Overall Yield |
|---|---|---|---|---|
| CP-0031535-13 | 34.9 g | 99.12% | 98.6% | 8.2% |

### Demonstration Batch (35 g) CP-0031535-13 Analysis:

| | Analytical | |
|---|---|---|
| Test Items | Method | Observation |
| Appearance | Visual | White crystalline solid powder |
| Identification | ¹H-NMR | Conform to structure |
| Purity | HPLC | 99.12% (a/a) |
| Enantiomeric purity | HPLC | 98.6% |
| Assay | ¹H NMR | 96.0-103.0% (w/w) |
| Residual solvents | GC | EtOH: 0.03%, MeCN: 0.02% |
| Water | K.F. | 0.147% (w/w) |
| Peak temperature | DSC | Complies with peak temperature of salt- |
| | | selection batch |
| Polymorph | XRPD | Complies with XRPD of salt-selection |
| | | batch |

### Validation Data

Additional confirmatory studies are included herein to validate the suitable salt form of crystalline ulodesine hemi-glutarate and its stability, thus re-confirming its suitability within the present pharmaceutical application.

Moreover, this data supports the rationale for purposive selection of the salt ulodesine hemi-glutarate as regards its use a medicament (or use in a pharmaceutically acceptable composition). Where other salts have failed in both stability and the potential for commercial upscaling, this specific salt choice provides an appropriate solution. In view of the re-confirmation of the relevant properties herein, it is asserted to be highly advantageous for that use, in particular, for use in the treatment and/or prevention for a variety of defined diseases and conditions, including those associated with a decline in tissue levels of the metabolite: NAD+.

In the present study, the free base of ulodesine and glutaric acid were used in several salt formations experiments using different solvent systems: EtOH, Acetone, EtOAc, THF, ACN/H2O (9:1) and DCM.

As was consistent with the findings in the disclosure WO2023001893, only one crystal form with high crystallinity was observed, this was named as Type A and was recrystallized from acetonitrile and water solvent mix ACN/H2O (9:1) as originally detailed in the method and experiments of WO2023001893. Other salt formation experiments only produced the free base or had low crystallinity.

Glutarate Type A was then scaled up to 500 mg to obtain sufficient material for evaluation using the following method: Glutarate Type A was re-prepared in ACN/H2O (9:1, v/v) at 500 mg scale.

Specifically, 499.34 mg of freebase Type A and 137.43 mg glutaric acid (charge ratio 0.55:1, acid/base) were weighed into a 20-mL vial, and 7 mL of corresponding solvent was added into the solids to form a suspension. After stirring magnetically at RT (~25 °C) for 1 day, the solids were isolated by vacuum filtration and vacuum dried at RT for 5 h. 600.67 mg of solids were obtained with the yield of 91.3%.

The product was the ulodesine Hemi glutarate speculated to be the monohydrate with high crystallinity. The water content by Karl Fischer titrator KF (wt %) 6.0 and stoichiometry (former/freebase) 0.5. The HPLC purity was 97.9 area %. The product was further characterised to confirm the following properties, summarised below and with reference to the figures:

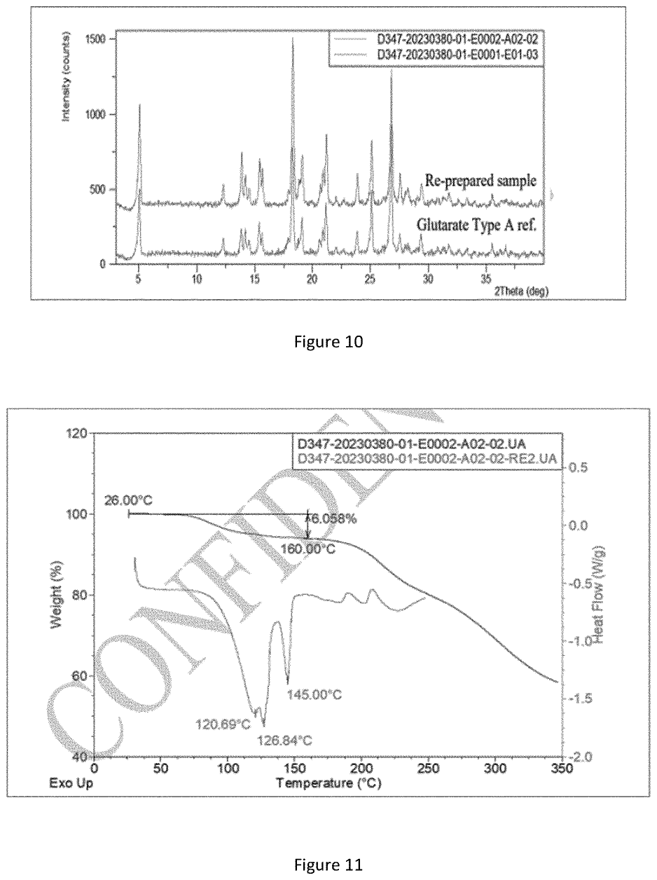

The XRPD diffractogram of the re-prepared sample (FIG. 10) indicated the obtained solids were glutarate Type A, as per the reference material.

The TGA result showed a weight loss of 6.1 wt % before 160.0 °C (theoretical 5.2% for one water), and the DSC result showed three endothermic peaks at 120.7 °C, 126.8 °C and 145.0 °C (peak) (FIG. 11).

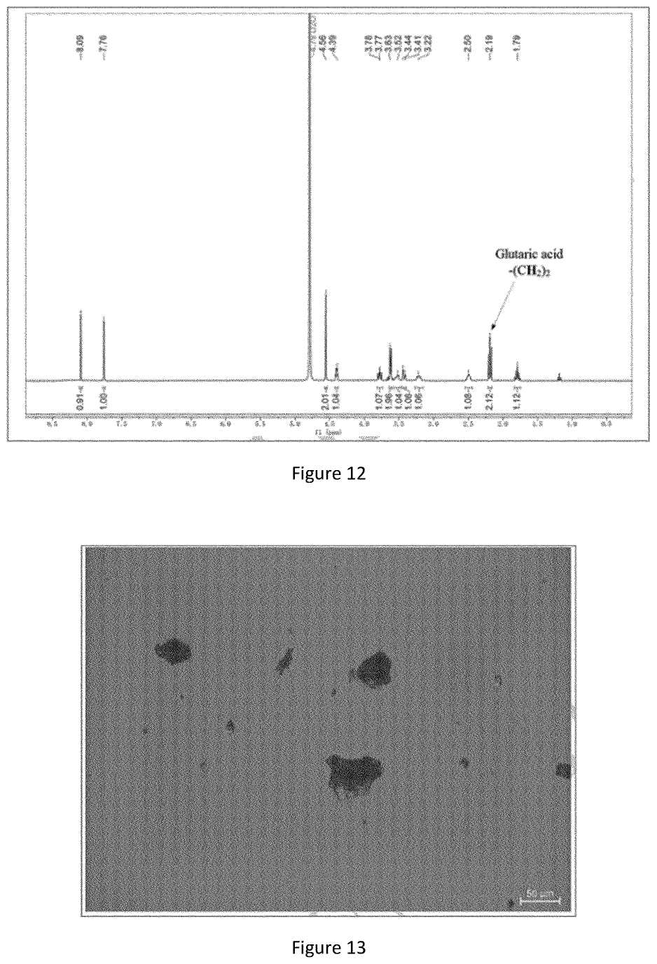

1H NMR spectrum (FIG. 12) of the re-prepared glutarate Type A showed the stoichiometry of glutaric acid/freebase was 0.5 with no residual ACN.

PLM image (FIG. 13) showed that the sample of Glutarate type A was small crystals and agglomerates consistent with the crystalline structure expected.

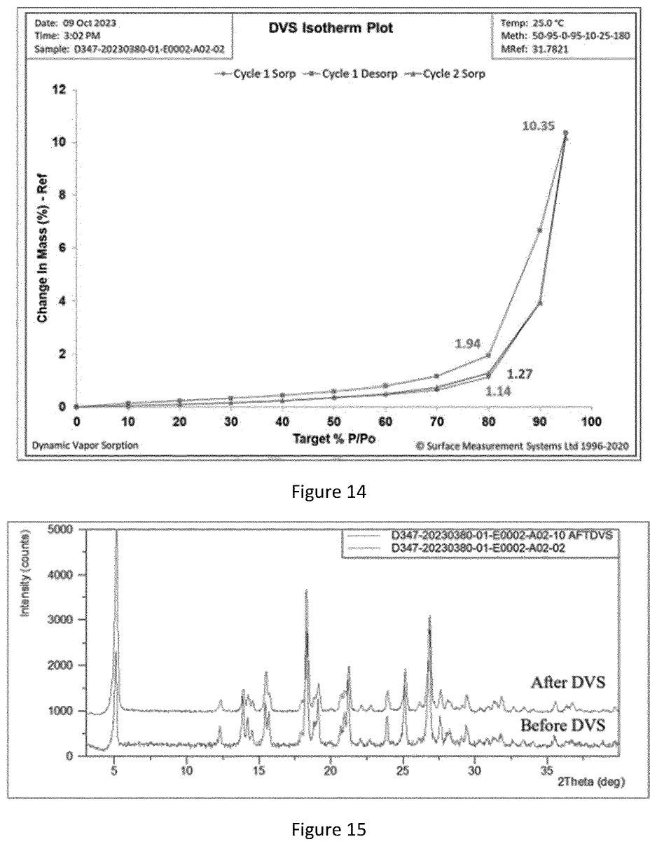

To understand the hygroscopicity of glutarate type A, DVS was employed to measure the mass change as a function of relative humidity at 25 °C (FIG. 14). Glutarate Type A showed a water uptake of 1.1% (calculated from 0~80% RH since no dehydration was observed at 0% RH) at 25 °C/80% RH, indicating glutarate Type A was slightly hygroscopic. At 25 °C/95% RH, the water uptake increased to 10.4%. XRPD results showed no form change after DVS (FIG. 15).

Kinetic solubility: (37 °C/24 h) over 16.8 mg/mL all (water/SGF/FaSSIF/FeSSIF) under pH value

### Physicochemical Stability

| | Initial | | 10-day | | 2-week | |
|---|---|---|---|---|---|---|
| Salt | purity | | Purity | Form | Purity | Form |
| (Batch No.) | (area %) | Condition | (area %) | change | (area %) | change |
| Glutarate Type A | 97.9 | 25 °C/60% RH | 97.7 | No | 97.8 | No |
| (D347-20230380-01- | | 40 °C/75% RH | 97.8 | No | 97.7 | No |
| E0002-A02-02 | | 60 °C | 97.6 | No | 97.3 | No |

(2-week, open conditions): no form change observed in all conditions for up to 2 weeks at (25 °C/60% RH, 40 °C/75% RH and 60 °C).

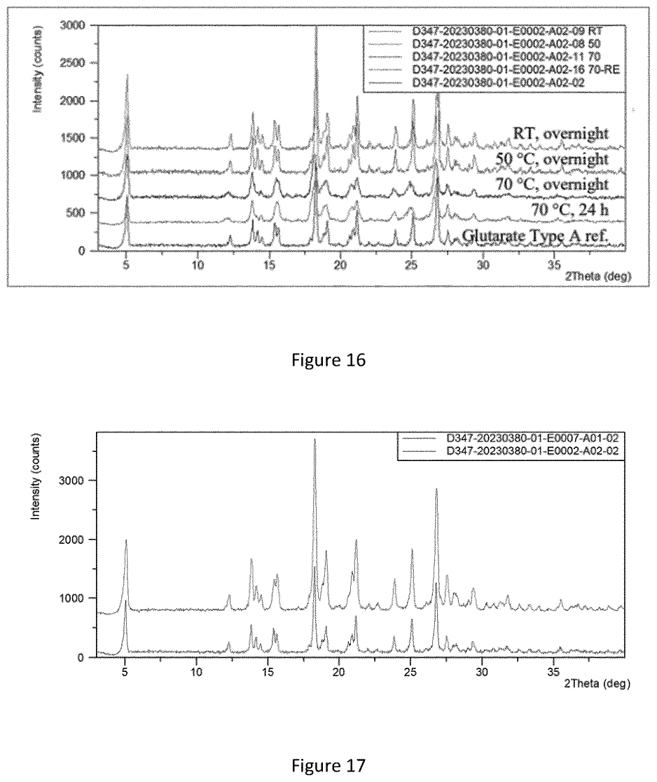

Drying stability (vacuum, RT/50/70 °C): vacuum drying overnight at RT and 50 °C, no change in crystallinity other than slight decrease at 70 °C (FIG. 16).

The term “mono” means the ratio of API:acid is 1:1, respectively, in the crystal structure of the salt of compound Ulodesine. The term “hemi” means the ratio of API:acid is 2:1, respectively, in the crystal structure of the salt of compound Ulodesine. The term “inert organic solvent” refers to a solvent that does not interfere chemically with the reaction.

The term “isostructural” is used to describe crystalline substances that have the same type of crystalline structure such as when a new molecular entity substitutes for another in a crystal structure without significantly disturbing the unit cell.

The “crystalline state” of the salt is determined by standard techniques and these are defined herein below. Crystals and their crystal structures are characterised using a number of techniques including single crystal X-ray crystallography, X-ray powder diffraction (XRPD) and differential scanning calorimetry (DSC).

The behaviour of the crystals under conditions of varying humidity can be analysed by gravimetric vapour sorption studies and by XRPD. These techniques help characterise the salts produced and confirm whether the product is optimised. In particular, X-ray crystallography involves the analysis and interpretation of the X-ray diffraction of single crystal. In an amorphous solid, the three dimensional structure that normally exists in a crystalline form does not exist and the positions of the molecules relative to one another in the amorphous form are essentially random.

During experimental formation, the hemi-glutarate salt is recrystallized from water and other organic solvents. The disclosure provides solvates formed by the incorporation of a non-toxic pharmaceutically acceptable solvent into the solid-state structure (e.g. crystal structure) of the compounds provided herein. Examples of such solvents include water, alcohols (such as ethanol, isopropanol and butanol) acetonitrile (ACN) and dimethylsulfoxide as described above. Thermogravimetric analysis (TGA), differential scanning calorimetry (DSC) and X-ray crystallography help determine if a solvate is formed. The solvates can be stoichiometric or non-stoichiometric solvates and may include hydrates such as hemihydrates, monohydrates and dihydrates. Alternatively, the resulting compound may be anhydrous (e.g. anhydrous crystalline form). However, here it has been speculated that the stable Type A form is a monohydrate.

The discussion herein is aided by the drawings in graphic form of various analysis techniques described below:

### Polarized Light Microscopy (PLM)

Photomicrographs were taken using a Nikon ECLIPSE Ci-POL polarized light microscope at room temperature equipped with eyepieces of 10× and objective of 5/20/50/100× magnification. Sample was dispersed with a drop on oil on a glass slide and a photomicrograph was taken.

### X-Ray Powder Diffraction (XRPD)

X-ray powder diffraction data was collected under ambient conditions on a Malvern Panalytical Aeris diffractometer. Flat a few milligrams of sample on a silicon sample holder and compact it by a glass slide with protection of a piece of weighting paper. An X-ray generator of 300 W was employed at 40 kV and 7.5 mA and was equipped with an X-ray tube of Cu (Kα), with the Kα2/Kα1 intensity ratio of 0.50 (1.54439 Å/1.5406 Å). Data were collected from 3° to 40° (2θ) at a scan speed of 0.14°/s with step size of 0.02° (2θ). X-ray powder diffraction data was collected under ambient conditions on a Malvern Panalytical Empyrean diffractometer. Flat a few milligrams of sample on a silicon sample holder and compact it by a glass slide with protection of a piece of weighting paper. An X-ray generator of 1800 W was employed at 45 kV and 40 mA and was equipped with an X-ray tube of Cu (Kα), with the Kα2/Kα1 intensity ratio of 0.50 (1.54439 Å/1.5406 Å). Data were collected from 3° to 40° (2θ) at a step size of 0.026° (2θ) with time per step of 49.7 s.

### Differential Scanning Calorimetry (DSC)

DSC is a thermoanalytical technique in which the difference in the amount of heat required to increase the temperature of a sample and reference are measured as a function of temperature. Differential scanning calorimetry was performed with a TA Discovery 2500 series DSC using a few milligrams of material in a Tzero aluminium pan sealed with a Tzero hermetic lid. Samples were analyzed using a heating rate of 10 °C per minute under 50 mL per minute of nitrogen flow. Heating Test: Differential scanning calorimetry was performed with a TA Discovery series DSC2500. Take a few milligrams of material into a Tzero aluminum pan and covered with a Tzero hermetic lid. Samples were analyzed from room temperature to target temperature and then cooled to room temperature, with heating rate of 10 °C per minute under 50 mL per minute of nitrogen flow.

### Thermogravimetric Analyses (TGA)

TGA is a type of testing that is performed on samples to determine changes in weight in relation to changes in temperature. Thermogravimetric analysis data were collected with a TA Discovery 5500 series TGA. Take a few milligrams of material to a Tzero aluminium pan and the sample were heated from room temperature to target temperature with a heating rate of 10 °C per minute under nitrogen protection (flow rate of 25 mL/min).

### Dynamic Vapour Sorption (DVS)

Dynamic vapour sorption was performed with ADVENTURE series DVS and Intrinsic-1 DVS at 25 °C under nitrogen blow. Approximately 30 milligrams of material were used. Anhydrate was analyzed using methods below: 0% RH to 95% RH to 0% RH at 10% RH (5% RH from 90% RH to 95% RH to 90% RH). After equilibrium at ambient conditions (50% RH), the hydrate sample was analyzed using method below: 1. Ambient relative humidity to 95% RH at 10% RH (5% from 90% RH to 95% RH). 2. 95% RH to 0% RH at 10% RH (5% from 95% RH to 90% RH). 3. 0% RH to 95% RH at 10% RH (5% RH from 90% RH to 95% RH).

The moisture sorption profile was generated at 25 °C using a DVS moisture balance flow system (Model Advantage 1.0) with the following conditions: sample size approximately 10 mg, drying 25 °C for 60 minutes, adsorption range 0% to 95% RH, desorption range 95% to 0% RH, and step interval 5%. The equilibrium criterion was <0.01% weight change in 5 minutes for a maximum of 120 minutes.

### Water Content (KF)

The water content data of the sample was collected using the Karl Fischer, from the Metrohm 870/803. Measured using the volumetric method, the titrant was HYDRAN/AL Composite 2 (FLUKA), the solvent was anhydrous methanol, and the stirring time was 400 s. The specific operation is to weigh 0.2 g of sample (accurate to 0.0001 g) and test the moisture content of the sample with the above KF water titrator.

### 1H NMR

1H NMR data was taken using Bruker AVANCE NEO 400 MHz in DMSO-d6 or D2O.

### High Performance Liquid Chromatography (HPLC)

SHIMADZU LC-20AD was employed to detect the chemical purity HPLC method for purity test

| Instrument | SHIMADZU LC-20AD | | |
|---|---|---|---|
| Column | XBridge ® Phenyl (4.6*150 mm 3.5 μm) | | |
| Column temp. | 30 °C | | |
| Mobile phase | A: 20 mM K₂HPO₄ in water B: ACN:MeOH = 1:1, v/v | | |
| Detection | 228 nm | | |
| wavelength | | | |
| Flow rate | 1.0 mL/min | | |
| Injection | 3 μL | | |
| Diluent | 10% ACN in H₂O | | |
| Conc. | 1.0 mg/mL | | |
| | Time (min) | A % | B % |
| Gradient | 0.00 | 98 | 2 |
| | 5.00 | 98 | 2 |
| | 21.00 | 90 | 10 |
| | 35.00 | 20 | 80 |
| | 40.00 | 20 | 80 |
| | 40.10 | 98 | 2 |
| | 45.00 | 98 | 2 |

### Polymorph Screen

To confirm stability and suitability of Ulodesine hemi-glutarate Type A, a polymorph screening was conducted to search for other potential crystal forms and identify the most suitable form to confirm the previous findings.

Ulodesine hemi-glutarate starting material E0007-A01-02) was first characterised by X-ray powder diffractometer (XRPD), thermogravimetric analysis (TGA), differential scanning calorimetry (DSC), polarized light microscopy (PLM), proton nuclear magnetic resonance (1H NMR) and high performance liquid chromatography (HPLC) by the methods previously described.

The characterization results indicated that the starting material was consistent with glutarate Type A, a monohydrate of hemi-glutarate.

The solubility of starting material (E0007-A01-02) was estimated at RT (~27 °C).

Specifically, approximate 2 mg of solid was added into an HPLC glass vial. The solvents below were then added stepwise (50/50/200/700 L) into the vials until the solids were dissolved or a total volume of 1.0 mL was reached.

The solubility data were used to guide the solvent selection in polymorph screening. Approximate solubility of starting material (E0007-A01-02) at RT:

| | Solubility | Solvent | Solubility |
|---|---|---|---|
| Solvent | (mg/mL) | (v/v) | (mg/mL) |
| MeOH | 1.9 < S < 6.5 | ACN | S < 1.9 |
| EtOH | S < 2.1 | DMSO | S > 39.6 |
| IPA | S < 1.8 | DMF | 2.3 < S < 7.6 |
| Acetone | S < 2.1 | DCM | S < 1.8 |
| MEK | S < 1.9 | n-Heptane | S < 2.0 |
| MIBK | S < 1.8 | H₂O | S > 36.0 |
| EtOAc | S < 2.1 | NMP | 1.9 < S < 6.3 |
| IPAc | S < 2.0 | DMAc | 2.0 < S < 6.5 |
| MTBE | S < 2.0 | DCM/MeOH (1:1) | S < 1.9 |
| CPME | S < 1.9 | Acetone/H₂O (9:1) | S < 1.9 |
| THF | S < 2.0 | THF/H₂O (9:1) | S < 2.0 |
| 2-MeTHF | S < 1.9 | EtOH/H₂O (9:1) | S < 2.0 |
| 1,4-Dioxane | S < 2.0 | EtOH/H₂O (4:1) | 2.1 < S < 6.9 |
| Toluene | S < 1.9 | EtOH/H₂O (2:1) | 22.6 < S < 45.2 |

| Abbre- | | Abbre- | |
|---|---|---|---|
| viation | Solvent | viation | Solvent |
| MeOH | Methanol | MEK | 2-Butanone |
| EtOH | Ethanol | ACN | Acetonitrile |
| IPA | Isopropanol | H₂O | Water |
| THF | Tetrahydrofuran | DCM | Dichloromethane |
| 2-MeTHF | 2-Methyltetrahydrofuran | DMF | N,N-Dimethylformamide |
| MTBE | Methyl tert-butyl ether | DMSO | Dimethyl sulfoxide |
| EtOAc | Ethyl acetate | CPME | Cyclopentyl methyl ether |
| IBAC | Isobutyl acetate | NMP | N-Methyl pyrrolidone |
| IPAc | Isopropyl acetate | DMAc | N,N-Dimethylacetamide |
| MIBK | Methyl isobutyl ketone | nBAc | n-Butyl acetate |
| NPA | n-Propanol | — | — |

Using glutarate Type A as starting material, a polymorph screening was conducted under 100 conditions through methods of anti-solvent addition, solid vapor diffusion, liquid vapor diffusion, slow evaporation, polymer-induced crystallization, temperature cycling, slurry at room temperature (RT)/50 °C and slow cooling.

The following is a summary of polymorph screening results for Ulodesine hemi-glutarate:

| | No. | |
|---|---|---|
| Method | of Exp. | Results |
| Anti-solvent addition | 19 | Glutarate Type A, freebase Type A/B |
| Solid vapor diffusion | 8 | Glutarate Type A |
| Liquid vapor | 11 | Glutarate Type A, freebase Type B, |
| diffusion | | amorphous + one peak, gel |
| Slow evaporation | 3 | Glutarate Type A, amorphous + one |
| | | peak, gel |
| Polymer-induced | 3 | Oil, amorphous + one peak, amorphous |
| crystallization | | |
| Temperature cycling | 12 | Glutarate Type A, glutarate Type C, |
| | | Oil |
| Slurry at RT | 18 | Glutarate Type A, glutarate Type C, |
| | | freebase Type C, amorphous |
| Slurry at 50 °C | 18 | Glutarate Type A, freebase Type B, |
| | | amorphous |
| Slow cooling | 8 | Glutarate Type A, amorphous, oil, gel |
| Total | 100 | Glutarate Type A/C, freebase Type |
| | | A/B/C, amorphous, amorphous + one |
| | | peak, gel, oil |

Two new crystal forms of hemi-glutarate (glutarate Type B and Type C) and two new forms of freebase (freebase Type B/C) were observed.

| | Wt. loss in TGA | DSC | Stoichiometry | Form change | Speculated |
|---|---|---|---|---|---|
| Crystal form | (%) | (peak, °C) | (acid/base) | after heating | form |
| Glutarate Type A | 5.7 | 117.8, 129.9, | 0.5 | Glutarate Type | Monohydrate |
| (01-E0007-A01-02) | (RT-160 °C) | 142.3 | | B (130 °C) | |
| Glutarate Type B | Glutarate Type B was obtained by heating glutarate Type | | | | Meta-stable |
| (01-E0007-A01-06) | A to 130 °C. After cooling back to RT, it quickly converted to | | | | form |
| | glutarate Type A, thus no characterization data were collected. | | | | |
| Glutarate Type C | 3.5 | 71.4, 105.7, | 0.5 | No | Channel |
| (02-E0008-A09-02) | (RT-140 °C) | 137.9, 155.9 | | | hydrate |

Characterization of hemi-glutarate crystal forms followed using the previously described analytical techniques with the addition of X-ray Powder Diffractometer at Variable Temperature (VT-XRPD):

X-ray powder diffraction data at variable temperature was collected at on a Malvern Panalytical Empyrean diffractometer. Flat a few milligrams of sample on a sample holder and compact it by a glass slide with protection of a piece of weighting paper. An X-ray generator of 1800 W was employed at 45 kV and 40 mA and was equipped with an X-ray tube of Cu (Kα), with the Kα2/Kα1 intensity ratio of 0.50 (1.54439 Å/1.5406 Å). Data were collected from 3° to 400 (2θ) at a step size of 0.026° (2θ) with time per step of 49.7 s.

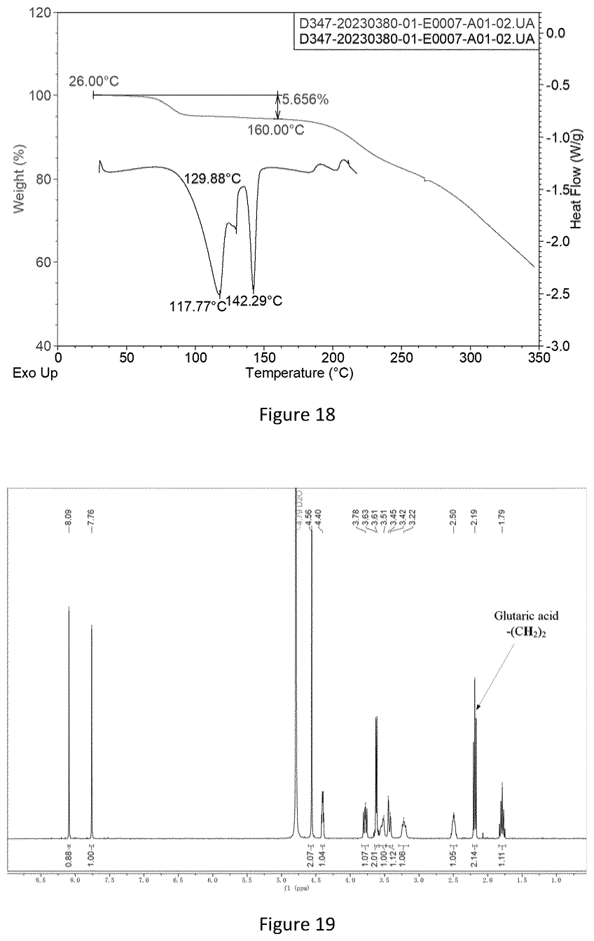

The Glutarate type A was characterised by XRPD/PLM/TGA/DSC/¹H NMR/HPLC. The XRPD diffractogram (FIG. 17) showed it was consistent with the hemi-glutarate type A starting material and consisted of small crystals and agglomerates. The TGA result showed a weight loss of 5.7 wt % before 160 °C (theoretical monohydrate is 5.2%), and the DSC data showed three endothermic peaks at 117.8 °C, 129.9 °C and 142.3 °C (peak) (FIG. 18). ¹H NMR spectrum (FIG. 19) showed the stoichiometry of glutaric acid/freebase was 0.5 with no residual ACN. The HPLC purity of the glutarate Type A was 98.0 area %.

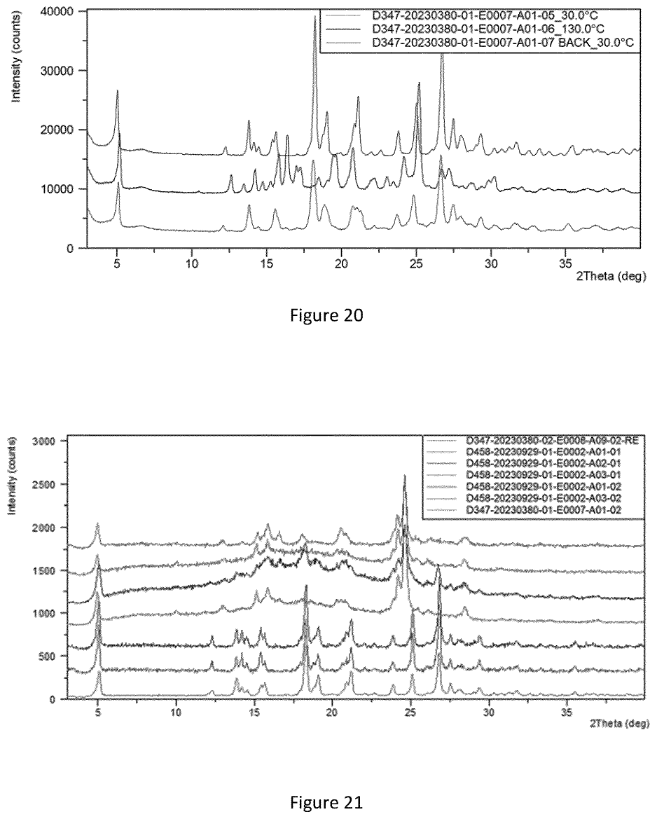

Glutarate Type B (E0007-A01-06) was only observed by heating glutarate Type A to 130 °C using X-ray powder diffractometer at variable temperature mode (VT-XRPD). After cooling back to RT, it quickly converted to glutarate Type A (FIG. 20). Based on the characterization results, glutarate Type B was a meta-stable form at ambient condition.

Glutarate Type C was obtained in anisole/MeOH (1:2, v/v) from temperature cycling slurry. The XRPD diffractogram showed no form change after drying at RT overnight. TGA/DSC results showed a weight loss of 3.5% (theoretical hemi-hydrate is 2.7%) up to 140 °C, and four endothermic peaks at 71.4 °C, 105.7 °C, 137.9 °C and 155.9 °C (peak). ¹H NMR spectrum showed the stoichiometry of acid/freebase was 0.5 with no residual solvents.

Glutarate Type C was further heated to 110 °C to study the thermal events, and the XRPD results showed no form change. The TGA/DSC results for the sample after heating showed a weight loss of 3.4% (theoretical hemi-hydrate is 2.7%) up to 140 °C, and two endothermic peaks at 61.4 °C and 156.4 °C (peak). Based on the characterization results, glutarate Type C was speculated as a channel hydrate (the water molecules of a channel hydrate lie next to other water molecules of adjoining unit cell, forming channels through the crystals).

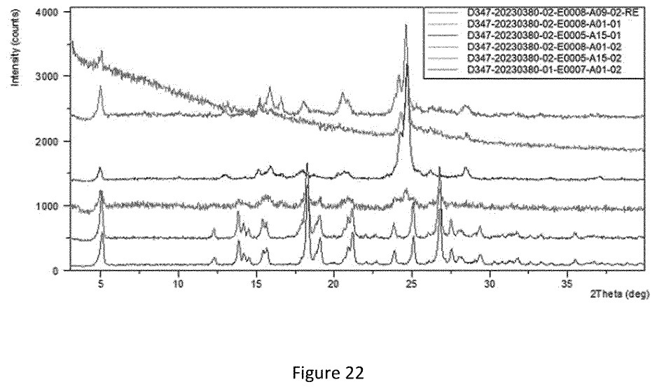

Type C was re-prepared at 100 mg in triplicate via temperature cycling slurry in anisole/MeOH (1:2, v/v). The XRPD results (see graphics in FIG. 21 and FIG. 22) showed two experiments generated glutarate Type C wet cakes, and the other one was glutarate Type A+C. However, both glutarate Type C wet cakes converted to glutarate Type A after drying under ambient condition for ~6 h (~20 °C/32% RH). Also, two batches of glutarate Type C wet cakes from screening (partially) converted to glutarate Type A after drying under ambient condition (~25 °C/30% RT). Based on the results glutarate Type C can easily transfer to glutarate Type A during drying under ambient condition, reflecting lower stability compared with glutarate Type A.

Based on the polymorph screening results, glutarate Type A showed good solid-state properties and stability and was thus confirmed to be the lead form for stable pharmaceutical development.

### TEST EXAMPLES

The following further test examples are set forth to illustrate certain aspects and features of the disclosure and should not be construed as limiting the full scope as defined by the claims appended hereto.

Example 1 demonstrates that dosing of PNP inhibitor ulodesine hemi glutarate in a diet induced obesity (DIO) mouse model, significantly reduces weight gain, serum cholesterol and blood glucose without any decrease in food consumption.

Example 2 demonstrates that dosing with ulodesine hemiglutarate in a DIO mouse model reduces serum cholesterol, reduces serum AST and ALT, reduces plasma glucose and insulin and improves insulin sensitivity. In addition, ulodesine treatment also demonstrated a significant decrease in inguinal white adipose tissue (iWAT)/Body weight (%), a decrease in fatty acid synthesis enzymes and an increase in thermogenic markers without any decrease in consumption of food.

Example 3 demonstrates the PNP inhibitor ulodesine hemi glutarate in a DIO mouse model significantly decreases total cholesterol and AST in liver.

Example 4 demonstrates that low and intermittent dosing of ulodesine hemi glutarate (2 mg/kg) does not significantly impact lymphocytes in a DIO mouse model.

### Example 1: Effect of Low and Intermittent Dosing of Ulodesine Hemi Glutarate in (DIO) Mouse Model

The aim of the preliminary study is to determine the effect of low and intermittent dosing of ulodesine hemi glutarate on the following parameters: weight gain, food intake, fasting blood glucose concentration and serum cholesterol. The study assesses ulodesine hemi glutarate efficacy on the listed parameters in the DIO mouse model of obesity and metabolic dysfunction.

Method: C57/BL6J mice were fed obesogenic diet D12492 (60 kcal % fat) from age 6 weeks. Eighteen week old mice (approximately 40 g) were either treated with vehicle (control) or ulodesine hemi glutarate at 2 mg/kg for 3 days a week (days 1, 2 and 3) given orally, over a 28 day period. On day −2 and on day 28, animals were fasted for 6 hr by withdrawing food only, and blood was collected for both blood glucose and cholesterol measurements.

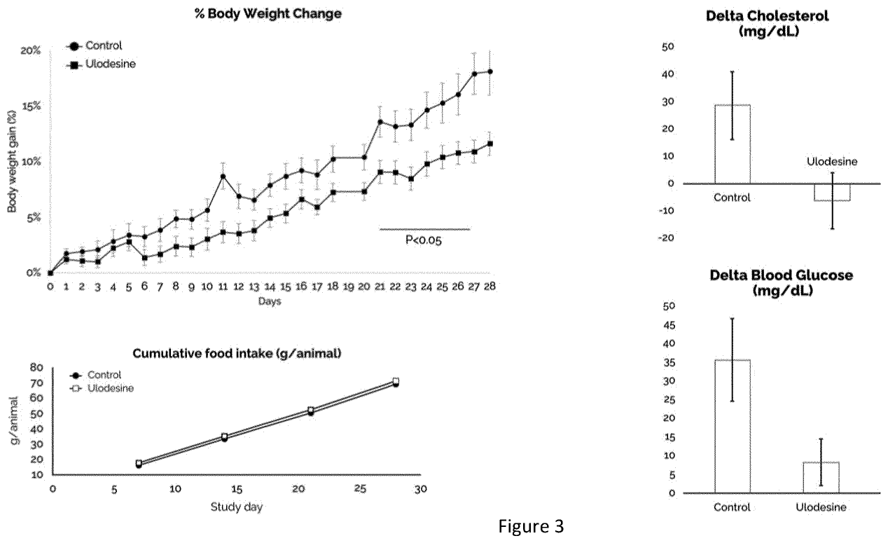

Results: As shown in FIG. 3, the ulodesine hemi glutarate treated group exhibited a significant decrease in weight gain compared to the control group. In addition to decrease in weight gain there was also decrease in blood glucose and serum cholesterol. The food intake of ulodesine hemi glutarate treated animals were similar to the control group. There were no signs of any toxicity or adverse effects of the drug.

### Example 2. Effect of Low and Intermittent Dosing of Ulodesine Hemi Glutarate in DIO Mouse Model

The aim of this study is to determine the effect of a low and intermittent dosing regime of ulodesine hemi glutarate on the following parameters: weight gain, food intake, fasting blood glucose concentration, serum insulin, serum cholesterol, plasma liver enzymes aspartate aminotransferase (AST) and alanine transaminase (ALT), inguinal white adipose tissue (iWAT), BAT; inosine in plasma and NAD+ in whole blood, and expression of lipogenesis (fatty acid synthesis) markers in iWAT and thermogenic markers in BAT. The study will assess ulodesine hemi glutarate efficacy on the listed parameters in the DIO mouse model of obesity and metabolic dysfunction.

Method: C57/BL6J mice were fed obesogenic diet D12492 (60 kcal % fat) from age 6 weeks. Nineteen week old mice (approximately 50 g) were either treated with vehicle (control) or ulodesine hemi glutarate at 2 mg/kg for 3 days a week (days 1, 2 and 3), given orally, over a 28 day period. On day minus 2 and on day 28, animals were fasted for 6 hr (only food withdrawn) and blood was collected for both blood glucose and cholesterol. On day 28 plasma liver enzymes (AST and ALT) and insulin were determined and additional plasma and whole blood was frozen and used for inosine and NAD+ measurement. BAT and iWAT was collected at the end of the study, weighed and quantitative PCR was performed for the lipogenesis markers Stearoyl CoA desaturase 1 (SCD-1) and fatty acid synthetase (FAS) and the thermogenesis marker uncoupling protein 1 (UCP-1). Inosine and NAD+ levels in stored plasma and whole blood respectively were determined by LC/MS/MS.

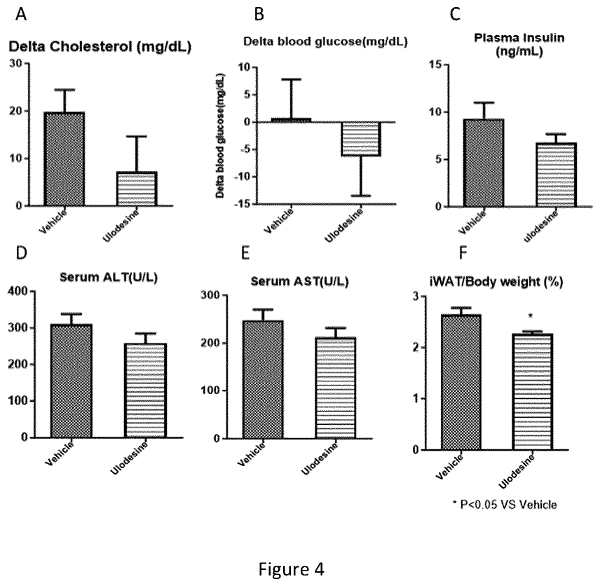

Results: The results are presented in FIG. 4. The ulodesine treated group showed decreases in blood glucose, plasma cholesterol, AST and ALT and insulin levels compared to the control group (FIG. 4A-E). There was significant decrease in iWAT/Body weight % in the ulodesine treated group compared to the control group (FIG. 4F).

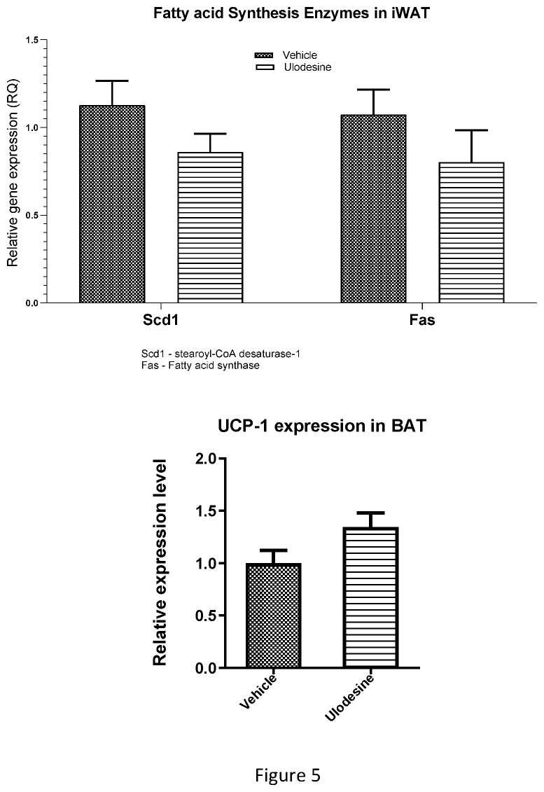

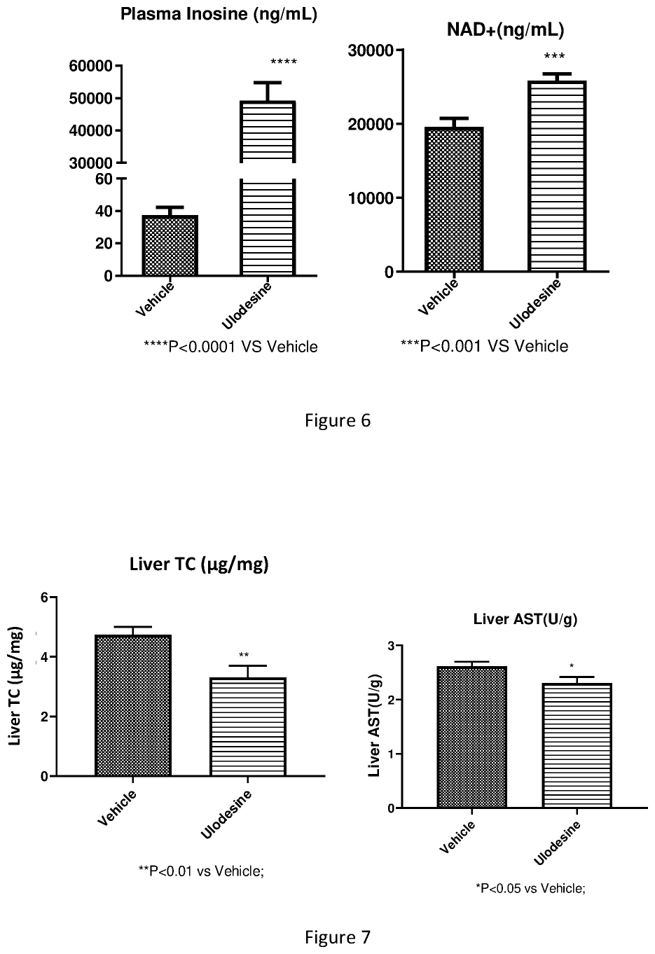

The ulodesine treated group demonstrated an increase in expression of the thermogenic marker, UCP-1 in the BAT tissue and decrease in lipogenesis markers (SCD-1 and FAS) in WAT tissue compared to the vehicle treated group (FIG. 5). As for example 1, the food intake of ulodesine hemi glutarate treated animals was similar to the control group. The decrease in weight gain in the ulodesine group was not significantly different from the control group unlike in the previous study. One possible reason is the starting weight of these animals was about 50 g in this study which is 25% more than the previous study and it may take longer treatment to realize significant decrease in weight gain. Both inosine and NAD+ were significantly elevated in plasma and whole blood respectively (FIG. 6).

These studies demonstrate that PNP inhibitor ulodesine hemi glutarate could potentially be used for the treatment of diseases associated with metabolic syndrome like obesity, fatty liver diseases and cardio-metabolic diseases. There were no signs of any toxicity or adverse effects of the drug.

### Example 3. Effect of Low and Intermittent Dosing of Ulodesine on Liver Parameters in a DIO Mouse Model

The aim of this study is to determine the effect of low and intermittent dosing of ulodesine hemi glutarate on the liver parameters: liver weight, total cholesterol, triglycerides, AST and ALT. The study assessed ulodesine hemi glutarate efficacy on the listed parameters in the DIO mouse model of obesity and metabolic dysfunction.

Method: C57/BL6J mice were fed obesogenic diet D12492 (60 kcal % fat) from age 6 weeks. Eighteen weeks old mice (approximately 40 g) were either treated with vehicle (control) or ulodesine hemi glutarate at 2 mg/kg for 3 days a week (days 1, 2 and 3) given orally for 8 weeks. At the end of eight weeks, the liver was removed and the following parameters were assessed: total cholesterol, triglycerides, AST and ALT.

Results: As shown in FIG. 7, the ulodesine treated group exhibited a significant decrease in total cholesterol (TC) and AST. Although there were decreases in triglycerides and ALT with treatment they did not achieve statistical significance.

This study supports the use of PNP inhibitors such as ulodesine for the treatment of fatty liver diseases.

### Example 4. Effect of Increasing Doses of Ulodesine on Lymphocytes Population in a DIO Mouse Model

The aim of this study is to determine the effect of increasing doses of ulodesine hemi glutarate on lymphocytes in whole blood.

Method: C57/BL6J mice were fed obesogenic diet D12492 (60 kcal % fat) from age 6 weeks. Eighteen weeks old mice (approximately 40 g) were either treated with vehicle (control) or ulodesine hemi glutarate at 1, 2, 5, and 10 mg/kg for only 3 days a week (days 1, 2 and 3) given orally for 4 weeks. At the end of four weeks, flow cytometry was used to enumerate total lymphocytes.

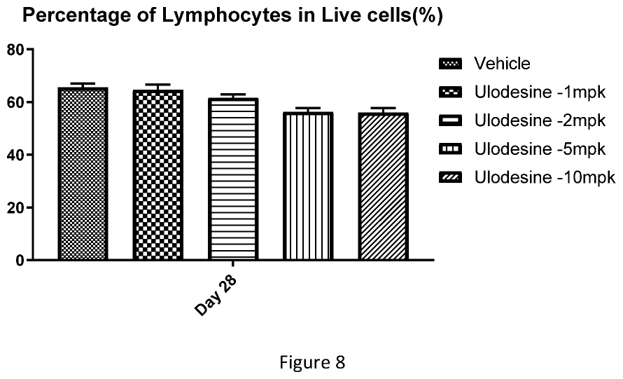

Results: As shown in FIG. 8, ulodesine hemi glutarate at 1 and 2 mg/kg does not impact the number of lymphocytes. Although, not significant at doses of 5 mg and 10 mg/kg, there appeared to be an observable decrease in lymphocytes. This study demonstrates that doses that improves various cardio metabolic parameters do not significantly impact lymphocytes.

1. A PNP inhibitor for use in the treatment or prevention of metabolic syndrome or a metabolic syndrome-induced disease or condition.

2. A PNP inhibitor for use according to claim 1 wherein treatment or prevention of metabolic syndrome includes treatment or prevention of at least one condition selected from increased blood pressure, high blood sugar, excess body fat, fatty liver and dyslipidemia.

3. A PNP inhibitor for use according to claim 1, wherein the metabolic syndrome-induced disease or condition is obesity or over weight.

4. A PNP inhibitor for use according to claim 1, wherein the metabolic syndrome induced disease or condition is selected from cardiovascular disease, and type 2 diabetes; optionally wherein the cardiovascular disease is selected from heart disease, peripheral vascular disease and stroke, or wherein the metabolic syndrome induced disease or condition is a fatty liver disease and optionally the fatty liver disease is selected from NAFLD and NASH.

5. (canceled)

6. The PNP inhibitor for use according to claim 1, wherein the PNP inhibitor is selected from a compound of the formula I, II and III: 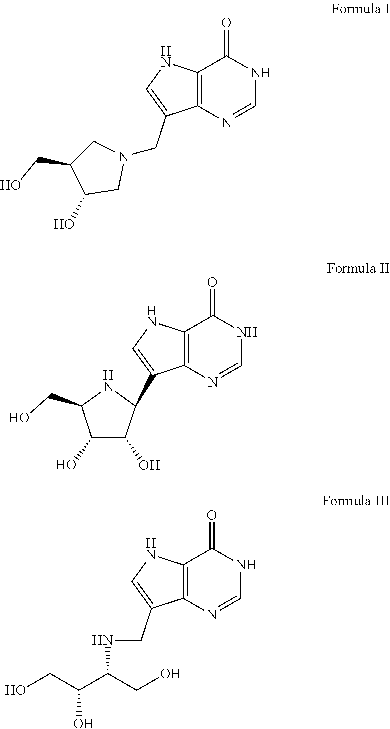 [SMILES: O=c1[nH]cnc2c(CN3C[C@H](CO)[C@@H](O)C3)c[nH]c12 ; O=c1[nH]cnc2c([C@@H]3N[C@H](CO)[C@@H](O)[C@H]3O)c[nH]c12 ; O=c1[nH]cnc2c(CN[C@H](CO)[C@H](O)CO)c[nH]c12]
or a pharmaceutically acceptable salt thereof.

7. The PNP inhibitor for use according to claim 5 which is a compound of the formula I 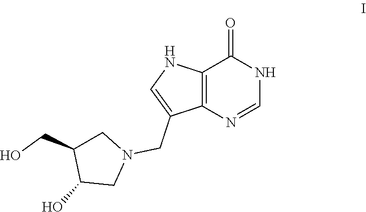 [SMILES: O=c1[nH]cnc2c(CN3C[C@H](CO)[C@@H](O)C3)c[nH]c12]
or a pharmaceutically acceptable salt thereof, optionally wherein the pharmaceutically acceptable salt is a hemi glutarate salt.

8. The PNP inhibitor for use according to claim 1, wherein the PNP inhibitor is provided at a dose of 1 mg to 1000 mg/day; optionally at a dose of 1 mg to 250 mg/day.

9. The PNP inhibitor for use according to claim 1, wherein the PNP inhibitor is provided at a dose of 1 mg to 150 mg; preferably at a dose of 10 mg to 40 mg

10. The PNP inhibitor for use according to claim 1, wherein the PNP inhibitor is provided in an intermittent dosing regime.

11. The PNP inhibitor for use according to claim 10, wherein the intermittent dosing regime comprises or consists of repeated cycles of treatment wherein one cycle of treatment comprises or consists of treatment for 1 to 6 days, followed by a break of at least 1 day; optionally wherein one cycle of treatment comprises or consists of treatment for a period of 2 to 4 days, followed by a break of at least 2 days.

12. The PNP inhibitor for use according to claim 8, wherein the dosage regimen results in a less than 20% reduction in blood lymphocyte population.

13. The PNP inhibitor for use according to claim 1, wherein the PNP inhibitor is provided in combination with at least one further active agent selected from ENT1 inhibitors, a GLP-1 agonists, anti-obesity agents and anti diabetic drugs.

14. A method of treating a subject suffering from metabolic syndrome or a metabolic syndrome induced disease, disorder or condition comprising administering to the subject a pharmaceutically effective amount of a PNP inhibitor.

15. The method of claim 14 wherein treatment of metabolic syndrome includes treatment or prevention of at least one condition selected from increased blood pressure, high blood sugar, excess body fat, fatty liver and dyslipidemia.

16. The method of claim 14, wherein the metabolic syndrome-induced disease or condition is obesity or over weight.

17. The method of claim 14, wherein the metabolic syndrome-induced disease or condition is selected from cardiovascular disease, and type 2 diabetes; optionally wherein the cardiovascular disease is selected from heart disease, peripheral vascular disease and stroke.

18. The method of claim 14, wherein the a metabolic syndrome-induced disease or condition is a fatty liver disease and optionally the fatty liver disease is selected from NAFLD and NASH.

19. A method of increasing the percentage of brown adipose tissue (BAT) to body weight in a subject in need thereof or improving the lipid profile in a subject in need thereof comprising administering to the subject a pharmaceutically effective amount of a PNP inhibitor.

20. (canceled)

21. The method according to claim 12, wherein the PNP inhibitor is provided in an intermittent dosing regime.

22. The method according to claim 14, wherein the PNP inhibitor is ulodesine, optionally provided as the hemi glutarate salt.
